(19) United States
(12) Patent Application Publication
(10) Pub. No.: US 2013/0204632 A1
(43) Pub. Date: Aug. 8, 2013

(54) Managing the Distribution of Drug Samples

(71) Applicants: Meredith Aull, New York, NY (US); Ioannis D. Papaioannou, New York, NY (US); John Del Giudice, New York, NY (US); Michael P. Pica, New York, NY (US)

(72) Inventors: Meredith Aull, New York, NY (US); Ioannis D. Papaioannou, New York, NY (US); John Del Giudice, New York, NY (US); Michael P. Pica, New York, NY (US)

(21) Appl. No.: 13/633,517

(22) Filed: Oct. 2, 2012

Related U.S. Application Data
(63) Continuation of application No. 13/189,949, filed on Jul. 25, 2011, now abandoned.
Application No. 13/189,949 is a continuation of application No. 12/266,287, filed on Nov. 6, 2008, now abandoned.

(51) Int. Cl. G06Q 10/08; G06Q 50/22

(52) U.S. Cl. CPC G06Q 10/087; G06Q 50/22
USPC 705/2

(57) ABSTRACT

Computer-based method and system that helps track and manage drug sample distribution, helps ensure drug samples are dispensed and used in a safe and efficient manner, and helps capture information about how drug samples are being distributed and used. The method includes storing inventory information for one or more drug samples received at a prescriber's office, presenting a user-selectable list of drugs available in the inventoried samples and, in response to a user's selection of one of the listed drugs, presenting patient-specific information regarding the selected drug's suitability for dispensing as a sample. The method also includes automatically updating the stored inventory information for the selected drug in response to an indication from the user that a quantity of the selected drug is being dispensed to a patient as a sample.

### BACKGROUND

The present disclosure relates to managing the distribution of drug samples to prescribers and patients. Prescribers may include, for example, physicians, doctors of osteopathy, physician assistants, nurse practitioners, dentists, and any other medical professionals licensed to prescribe medications.

Efficient delivery of sample medications to prescribers can be a highly effective marketing tool. The Prescription Drug Marketing Act (PDMA) of 1987 mandates that pharmaceutical manufacturers follow specific rules regarding the distribution of drug samples, or risk substantial fines and even being barred from distributing samples.

Benefits of drug sample distribution include free access for patients who could not otherwise afford the drug, trials to assess the benefits and risks of a new drug in an individual patient. Also, prescribers can observe the effects of the new medication in their specific patient population.

Concerns exist about the distribution of drug samples at least in part because pharmacists generally are not involved in such distributions.

### SUMMARY

The present disclosure relates to managing the distribution of drug samples to patients. More particularly, a computer-based method and system helps track and manage drug sample distribution, helps ensure that drug samples are dispensed and used in a safe and appropriate manner and helps capture information about how the drug samples are being used.

In one aspect, a computer-based method includes storing inventory information for one or more drug samples received at a prescriber's office, presenting a list of drugs available in the inventoried samples and automatically updating the stored inventory information for a selected drug in response to an indication from a user that a quantity of the selected drug is being dispensed to a patient as a sample.

In some implementations, the method includes enabling a pharmaceutical manufacturer to enter details describing a drug sample being sent to the prescriber's office, enabling the prescriber to confirm receipt of the drug sample and, in response to the prescriber's confirmation, updating the stored inventory information for the received drug sample. In response to the user's selection of one of the listed drugs, the method may include, presenting patient-specific information regarding the selected drug's suitability for dispensing as a sample. The patient-specific information regarding the selected drug's suitability for dispensing as a sample includes information can include, for example, whether the patient has allergies to the selected drug, whether the selected drug has the potential to cause adverse side-effects to the patient by interacting with other drugs that have been prescribed to the patient, whether the selected drug is contraindicative to a disease that the patient has been diagnosed with and whether administration of the selected drug might represent a therapeutic duplication of another drug that the patient is taking. The patient-specific information also may include whether the selected drug would be covered by the patient's insurance if the selected drug were prescribed to the patient.

Certain embodiments include presenting a user-selectable list of directions for use of the selected drug, wherein the directions have been approved by the Food and Drug Administration for use of the selected drug and generating, in response to the user selecting one of the directions for use, a label that includes the selected directions for use and patient specific information (e.g., name, address, etc.).

Some implementations of the method include, in response to a user prompt, printing educational material associated with the selected drug. The educational material can include how to use the selected drug, the selected drug's potential side effects and/or the selected drug's monitoring parameters.

According to certain embodiments, the method includes prompting the user to specify a reason why the selected drug is being dispensed as a sample to the patient. This prompting can include, for example, presenting a user-selectable list of reasons for dispensing the selected drug as a sample to the patient. The list of possible reasons can include, for example, to conduct a trial of the selected drug, to manage an acute condition with the selected drug, to respond to the patient's financial limitations and to respond to the patient's request for the selected drug.

After a prescriber has dispensed one of the drugs as a sample to the patient and subsequently prescribed a different one of the drugs to the patient, the method can include tracking what is later prescribed and enabling a manufacturer of the drug dispensed as a sample to access information related to what is later prescribed.

Certain implementations include prompting the user to enter demographic data for the patient to whom the drug sample is being dispensed (usually performed by office staff and some information can be retrieved through a “ping” to RxHub); and enabling a manufacturer of the drug sample being dispensed to access the demographic data associated with the patient in a manner that protects the patient's privacy.

The method also can include generating, in response to a user prompt, a list of patients to whom a particular one of the drugs has been dispensed as a sample, based on drug name, expiration date or lot number.

The stored inventory information includes: drug names, strength of sample drug available quantities of the drugs at the prescriber's office and the drugs' lot numbers and expiration dates. In some implementations, the method includes automatically updating the stored inventory information for the selected drug in response to an indication from the user that a quantity of the selected drug is being dispensed to the patient as a sample. The method also can include automatically identifying when the inventory of one or more of the drug samples has reached or dropped below a pre-defined minimum value. The method also can include automatically notifying a manufacturer of the drug sample whose inventory has reached or dropped below the pre-defined minimum value that additional inventory should be sent to the prescriber's office.

In certain embodiments, the method includes enabling the user to generate a list of drug samples in the prescriber office's inventory based on drug name, expiration date and lot number.

In another aspect, a computer-based method includes storing inventory information for one or more drug samples received at a prescriber's office, presenting a user-selectable list of drugs available in the inventoried samples and, in response to a user's selection of one of the listed drugs, presenting patient-specific information regarding the selected drug's suitability for dispensing as a sample.

In some implementations, the patient-specific information regarding the selected drug's suitability for dispensing as a sample includes: whether the patient has allergies to the selected drug, whether the selected drug has the potential to cause adverse side-effects to the patient by interacting with other drugs that have been prescribed to the patient, whether the selected drug is contraindicative to a disease that the patient has been diagnosed with, whether administration of the selected drug might represent a therapeutic duplication of another drug that the patient is taking and whether the selected drug would be covered by the patient's insurance if the selected drug were prescribed to the patient.

Certain embodiments include presenting a user-selectable list of FDA approved directions for use of the selected drug, and generating, in response to the user selecting one of the directions for use, a label that includes the selected directions for use and patient specific information (e.g., name, address, etc.).

According to some implementations, the user is prompted to specify a reason why the selected drug is being dispensed as a sample to the patient. Prompting can include, for example, presenting a user-selectable list of reasons for dispensing the selected drug as a sample to the patient. The list of possible reasons can include, for example, to conduct a trial of the selected drug, to manage an acute condition with the selected drug, to respond to the patient's financial limitations and/or to respond to the patient's request for the selected drug.

In some embodiments, after a prescriber has dispensed one of the drugs as a sample to the patient and subsequently prescribed a different one of the drugs to the patient, the method includes generating one or more reports that the manufacturer of the drug dispensed can use to assess prescribing trends. In some implementations, the system (e.g., the host) captures relevant information to generate the reports based on the physician's interactions with the system. The system organizes the relevant information in a useful format and presents the organized information in a manner that is accessible by the drug manufacturer. In one example, the report indicates: how often a distributed drug sample has led to a prescription subsequently being written for that drug and how often a distributed drug sample did not lead to a prescription subsequently being written for that drug. The report also may (or may not) include some indication as to why the drug sample distribution did not lead to a prescription being written (e.g., co-pay too high, sample did not work as expected, etc.).

Some implementations include prompting the user to enter demographic data for the patient to whom the drug sample is being dispensed (usually performed by office staff and some information can be retrieved through a “ping” to RxHub) and enabling a manufacturer of the drug sample being dispensed to access the demographic data associated with the patient in a manner that protects the patient's privacy.

In certain embodiments, the method of claim 18 includes generating, in response to a user prompt, a list of patients to whom a particular one of the drugs has been dispensed as a sample, based on drug name, strength of sample drug, expiration date or lot number.

Some embodiments include automatically updating the stored inventory information for the selected drug in response to an indication from the user that a quantity of the selected drug is being dispensed to the patient as a sample, automatically identifying when the inventory of one or more of the drug samples has dropped below a pre-defined minimum value and automatically notifying a manufacturer of the drug sample whose inventory has dropped below the pre-defined minimum value that additional inventory should be sent to the prescriber's office.

In another aspect, a system includes a first user interface device and one or more computers operable to interact with the user interface device and to: store inventory information for one or more drug samples received at a prescriber's office, present a list of drugs available in the inventoried samples and automatically update the stored inventory information for a selected drug in response to an indication from a user that a quantity of the selected drug is being dispensed to a patient as a sample.

The one or more computers can include, for example, one or more databases of patients' formulary and medical history. The patient-specific information regarding the selected drug's suitability for dispensing as a sample can be obtained from the database and can include information, such as, whether the patient has allergies to the selected drug, whether the selected drug has the potential to cause adverse side-effects to the patient by interacting with other drugs that have been prescribed to the patient, whether the selected drug is contraindicative to a disease that the patient has been diagnosed with, whether administration of the selected drug might represent a therapeutic duplication of another drug that the patient is taking and whether the selected drug would be covered by the patient's insurance if the selected drug were prescribed to the patient.

In some implementations, the one or more computers are operable to present, at the first user interface device, a user-selectable list of directions for use of the selected drug, wherein the directions presented are directions that have been approved by the Food and Drug Administration for use of the selected drug, generate, in response to the user selecting one of the directions for use, a label that includes the selected directions for use and patient specific information and enable the user at the first user interface device to print educational material associated with the selected drug. The educational material can include material such as how to use the selected drug, the selected drug's potential side effects and the selected drug's monitoring parameters.

In certain embodiments, the one or more computers are operable to prompt the user at the first user interface device to specify a reason why the selected drug is being dispensed as a sample to the patient. Prompting the user at the user interface to specify the reason for dispensing the selected drug as a sample to the patient can include presenting a user-selectable list of reasons for dispensing the selected drug as a sample to the patient. The list of reasons can include, for example, to conduct a trial of the selected drug, to manage an acute condition with the selected drug, to respond to the patient's financial limitations and to respond to the patient's request for the selected drug.

In some embodiments, after a prescriber has dispensed one of the drugs as a sample to the patient and subsequently prescribed a different one of the drugs to the patient, the one or more computers are operable to generate one or more reports that the manufacturer of the drug dispensed can use to assess prescribing trends.

According to some embodiments, the system includes a second user interface device. In such embodiments, the one or more computers can be further operable to prompt the user at the first user interface device to enter demographic data for the patient to whom the drug sample is being dispensed and enable a manufacturer of the drug sample being dispensed to access at the second user interface device the demographic data associated with the patient in a manner that protects the patient's privacy.

Certain implementations include a second user interface device at a location that corresponds to a manufacturer of the selected drug. In those implementations, the one or more computers are further operable to automatically update the stored inventory information for the selected drug in response to an indication from the user that a quantity of the selected drug is being dispensed to the patient as a sample, automatically identify when the inventory of one or more of the drug samples at the prescriber's office has dropped below a pre-defined minimum value and automatically notify the manufacturer that additional inventory should be sent to the prescriber's office.

In yet another aspect, a system includes a first user interface device and one or more computers operable to interact with the user interface device and to store inventory information for one or more drug samples received at a prescriber's office, present at the user interface device a user-selectable list of drugs available in the inventoried samples and, in response to a user's selection of one of the listed drugs, presenting at the user interface device patient-specific information regarding the selected drug's suitability for dispensing as a sample.

In some implementations, the system includes a second user interface device at a location that corresponds to a manufacturer of the selected drug. The one or more computers are further operable to automatically update the stored inventory information for the selected drug in response to an indication from the user that a quantity of the selected drug is being dispensed to the patient as a sample, automatically identify when the inventory of one or more of the drug samples at the prescriber's office has dropped below a pre-defined minimum value and automatically notify the manufacturer that additional inventory should be sent to the prescriber's office.

In certain embodiments, the one or more computers include one or more databases of patients' formulary and medical history. The patient-specific information regarding the selected drug's suitability for dispensing as a sample can be obtained from the one or more databases and may include: whether the patient has allergies to the selected drug, whether the selected drug has the potential to cause adverse side-effects to the patient by interacting with other drugs that have been prescribed to the patient, whether the selected drug is contraindicative to a disease that the patient has been diagnosed with, whether administration of the selected drug might represent a therapeutic duplication of another drug that the patient is taking and whether the selected drug would be covered by the patient's insurance if the selected drug were prescribed to the patient.

In some implementations, the one or more computers are further operable to present, at the first user interface device, a user-selectable list of directions for use of the selected drug, wherein the directions presented are directions that have been approved by the Food and Drug Administration for use of the selected drug, generate, in response to the user selecting one of the directions for use, a label that includes the selected directions for use and patient specific information and enable the user at the first user interface device to print educational material associated with the selected drug. The educational material can include material, such as how to use the selected drug, the selected drug's potential side effects and the selected drug's monitoring parameters.

According to certain embodiments, the one or more computers are further operable to prompt the user at the first user interface device to specify a reason why the selected drug is being dispensed as a sample to the patient. Prompting the user at the user interface to specify the reason for dispensing the selected drug as a sample to the patient can include presenting a user-selectable list of reasons for dispensing the selected drug as a sample to the patient. The list of reasons can include, for example, to conduct a trial of the selected drug, to manage an acute condition with the selected drug, to respond to the patient's financial limitations and to respond to the patient's request for the selected drug.

In some implementations, the system includes a second user interface device at a drug manufacturer's facility. In such implementations, after a prescriber has dispensed one of the drugs as a sample to the patient and subsequently prescribed a different one of the drugs to the patient, the one or more computers can generate one or more reports that the manufacturer of the drug dispensed can view at the second user interface device to assess prescribing trends.

Certain embodiments of the system include a second user interface device and the one or more computers are further operable to prompt the user at the first user interface device to enter demographic data for the patient to whom the drug sample is being dispensed and enable a manufacturer of the drug sample being dispensed to access at the second user interface device the demographic data associated with the patient in a manner that protects the patient's privacy.

The one or more computers can include one or more databases of patients' formulary and medical history, and the patient-specific information regarding the selected drug's suitability for dispensing as a sample can be obtained from or derived from data in the one or more databases. The information can include, for example, whether the patient has allergies to the selected drug, whether the selected drug has the potential to cause adverse side-effects to the patient by interacting with other drugs that have been prescribed to the patient, whether the selected drug is contraindicative to a disease that the patient has been diagnosed with and whether administration of the selected drug might represent a therapeutic duplication of another drug that the patient is taking.

In yet another aspect, a computer program product, encoded on a computer-readable medium, operable to cause a data processing apparatus to perform operations including: storing inventory information for one or more drug samples received at a prescriber's office, presenting a list of drugs available in the inventoried samples and automatically updating the stored inventory information for a selected drug in response to an indication from a user that a quantity of the selected drug is being dispensed to a patient as a sample.

In still another aspect, a computer program product, encoded on a computer-readable medium, operable to cause a data processing apparatus to perform operations including storing inventory information for one or more drug samples received at a prescriber's office; presenting a user-selectable list of drugs available in the inventoried samples; and in response to a user's selection of one of the listed drugs, presenting patient-specific information regarding the selected drug's suitability for dispensing as a sample.

Particular embodiments of the subject matter described in this specification can be implemented to realize one or more of the following advantages.

For example, certain embodiments provide screening for potential allergies, drug interaction problems and other important patient-related safety issues. Certain embodiments provide the ability to print a patient-specific medication label and drug information leaflet (similar to that which a patient would normally receive from a pharmacist).

Additionally, some implementations facilitate tracking drug samples so that the drugs are not diverted from their intended use. Pharmaceutical manufacturers can benefit from a reduction in fraud and abuse associated with drug sample distribution.

Moreover, pharmaceutical manufacturers can learn what happens to the drug samples once they reach the prescribers (e.g., does it go to a patient or does it sit on a shelf until it expires?). This type of information can be helpful to manufacturers in deciding how to market drugs and how to invest in research and development. In addition, certain implementations facilitate gathering real-time marketing data by tracking reasons why a sample was dispensed (e.g., acute therapy, trial of new medication in a specific patient, lack of insurance coverage, request from patient as result of direct-to-consumer advertising campaign).

The systems and techniques can facilitate the management of a prescriber's inventory of drug samples. They can facilitate recalls and distribution of black box warnings. In addition, expiration date reports can be generated to allow prescribers to easily and quickly generate list of sample medications that are nearing their expiration date, allowing removal from inventory rather than having to perform monthly manual checks of the inventory.

A variety of data about drug sample distribution can be shared among interested parties in a manner that maintains the confidentiality of patients.

Certain implementations help enable pharmaceutical manufacturers to effectively and efficiently comply with PDMA.

The pharmaceutical industry may be able to assess easily whether their drug samples lead to an increase in prescribing volume of the sampled agent. Some implementations capture the intent of prescribers in prescribing drugs. The implementations may track medication that a prescriber initially prescribes and then track the reasons why that medication may have been changed (e.g., safety issue, lack of insurance coverage).

In some implementations, the prescriber can see if the drug he/she wishes to give as a sample will be covered by the patient's insurance if they choose to later prescribe the medication.

Certain embodiments include bar coding of sample medications and interface with medication dispensing cabinet that can improve ease-of-use and enhance security of sample medications, if desired.

The details of one or more embodiments of the invention are set forth in the accompanying drawings and the description below. Other features, aspects, and advantages of the invention will become apparent from the description, the drawings and the claims.

### BRIEF DESCRIPTION OF THE DRAWINGS

FIG. 1 is schematic block diagram of various parties interacting with a computer-system that facilitates the distribution of drug samples by the illustrated parties.

FIG. 2 is a schematic block diagram of a computer system adapted to facilitate the distribution of drug samples.

FIG. 3 is a flowchart of a method of managing various aspects of a drug sample distribution process.

FIGS. 4-11 illustrate examples of screenshots presented to a prescriber interacting with the system.

FIG. 12 is a table indicating some of the system data that various parties can access.

### DETAILED DESCRIPTION

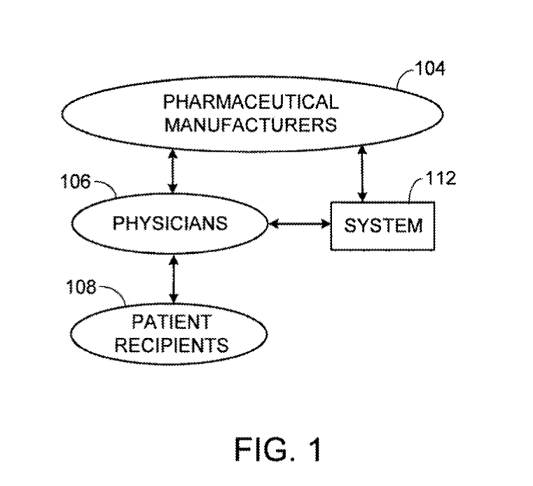

FIG. 1 is a block diagram showing various parties interacting with a computer-based system 112 that facilitates the management of drug sample distribution.

In general, the pharmaceutical manufacturers 104 distribute drug samples to the prescribers 106, who then distribute the drug samples to the patient recipients 108. In some implementations, a manufacturer's sales representative (not shown) may act on behalf of the manufacturer in distributing the drug samples to the prescribers. Drug samples may be distributed to patients for a variety of reasons including, for example, to help manage acute condition that the patient is suffering from, to conduct a trial of the drug in a specific patient, because the patient cannot otherwise afford the drug or because a patient has requested the drug. The system 112 helps manage this process.

In the illustrated implementation, the system 112 collects various information about patients and the drug samples being dispensed to the patients. It processes, shares and acts on this information to help the prescribers 106 and pharmaceutical manufacturers 104 more effectively distribute the drug samples. The system maintains this information in one or more databases that receive the information from a variety of sources. The sources can include, for example, prescribers, pharmaceutical manufacturers and remote sources, such as the National Patient Health Information Network™ provided by RxHub, LLC. The National Patient Health Information Network™ provides secure access over the Internet to medical information pertaining to over a hundred million people with prescription coverage in the United States. The information includes prescription eligibility, benefit information, formulary and medication histories and other information about consenting patients.

The system 112 is adapted to share the information it collects with the pharmaceutical manufacturers 104, the prescribers 106 and with the patient information database in a user-friendly manner, that protects the patients' privacy and helps the various parties involved in managing the distribution of drug samples to do so in a safe, effective and efficient manner. In a particular implementation, the system 112 also helps automate the maintenance of a drug sample inventory at the prescribers' offices; it helps ensure that patients receive clear and safe instructions for using the drug samples; it provides access to data that helps the parties involved understand how the drug samples are being used and whether their use is leading to the sampled drugs ultimately being prescribed. The system 112 may include a number of other functionalities and advantages as well.

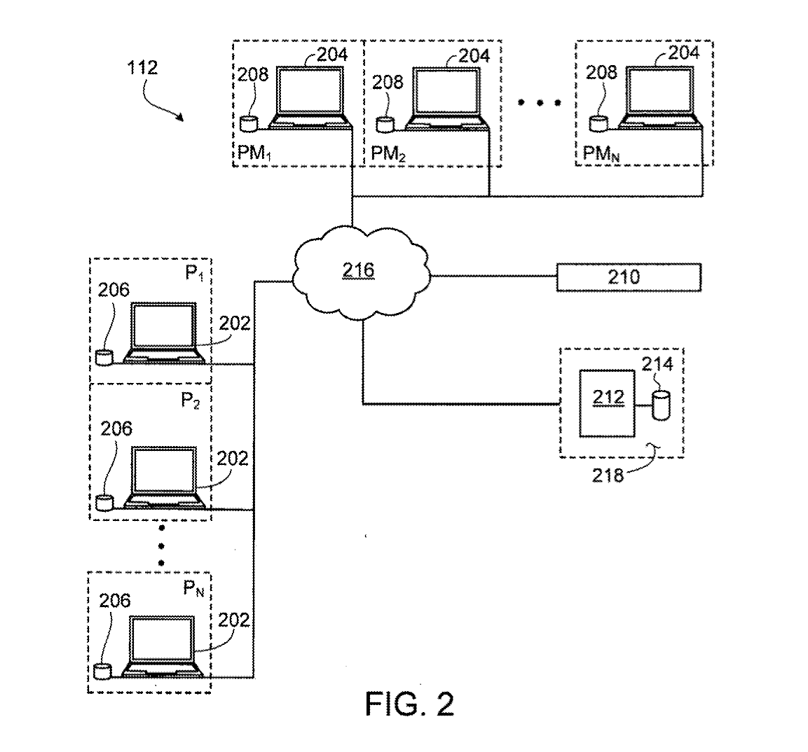

FIG. 2 is a detailed schematic diagram of an example of the system 112. The illustrated system 112 includes a first user interface devices 202 at each participating prescriber's office P1, P2, . . . PN. The first user interface devices (e.g., personal computers) 202 enable prescribers and their staff to access and interact with the system 112. As shown, each first user interface device 202 is connected to a computer network that is adapted to implement, in conjunction with the first user interface devices 202, the functionality of the system 112 and the various techniques disclosed herein.

In the illustrated implementation, the computer network includes a local memory storage device 206 in each of the participating prescribers' offices P1, P2, . . . PN. The local memory storage devices 206 typically store patient-specific data for patients of the prescriber in whose office the device is located. Second user interface devices 204 (e.g., personal computers) and associated local memory storage devices 208 are provided at each participating pharmaceutical manufacturer (or manufacturer's sales representative) location PM1, PM2, . . . PMN. The second user interface devices 204 enable pharmaceutical manufacturers to access and interact with the system 112. Each local memory storage device 208 typically stores data that relates to the drug samples that the pharmaceutical manufacturer has distributed.

A host 218 includes a server 212 and database 214 coupled to one another. The database 214 stores various data to support the system's 112 operations and functionality. The server implements various aspects of the system's 112 operations and functionality. In some implementations, the host hosts a website where various aspects of the operations and functionality described herein can be implemented.

A patient information database 210 (e.g., Surescript-Rx Hub and/or FirstDataBank) also is shown. In a typical implementation, the patient information database 210 provides the host with patient-specific data about medication history and prescription coverage. In some implementations, the patient information database 210 includes more than one database connected across a network. In some implementations, the patient information database can include information about patients' allergies, diagnosed diseases and/or conditions. Typically, the prescriber and/or the prescriber's office staff locally enters information about patients' allergies and diagnosed diseases and/or conditions.

In the illustrated implementation the various components are coupled to one another as shown over a wireless communication network 216 (e.g., the Internet).

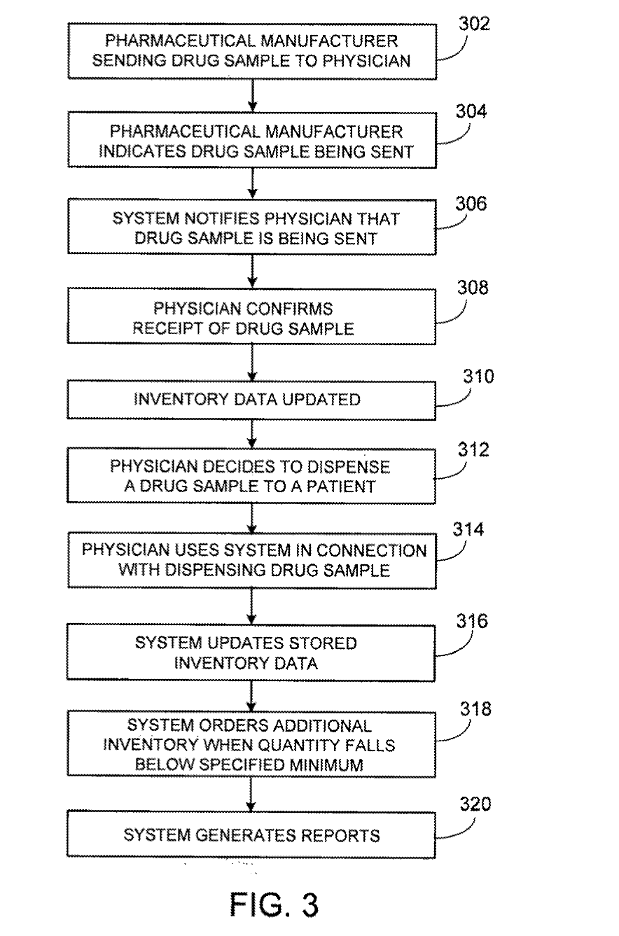

FIG. 3 is a flowchart showing an example of how the various parties involved in managing the distribution of drug samples conduct the distribution and interact with each other and with the system 112 of FIG. 2.

In the illustrated implementation, one of the participating pharmaceutical manufacturers 104 sends a drug sample to one of the participating prescribers 106 (block 302). As the drug sample is being sent, the pharmaceutical manufacturer enters an indication into second user interface device 204 that the drug sample is being sent (block 304). In a typical implementation, the pharmaceutical manufacturer would enter this indication on a secure website that prompts the pharmaceutical manufacturer to enter various information about the drug sample being sent. Such information can include, for example, the name(s) and strength(s) of the drug(s) being sent, the quantity(ies) of the drugs) being sent, the lot number(s) and expiration date(s) for the drug(s) being sent. The pharmaceutical manufacturer also may specify the method of delivery being used.

In some implementations, the drug samples may have a machine-readable data label coupled to the drug samples being sent. The label can be affixed using an adhesive, for example, to the drug sample's package or to a container of drug sample packages. The machine-readable data can include various information about the drug sample shipment including, for example, the drug names and strengths, quantities, lot numbers and expiration dates. Suitable-machine-readable labels are bar-codes and the like. If a bar code or other machine-readable label is affixed to the drug samples being sent, the pharmaceutical manufacturer may be able simply to scan the bar code to enter data about the drug samples being shipped into the system.

In response to the pharmaceutical manufacturer indicating that the drug sample is being sent, the system 112 electronically notifies the destination prescriber's office to expect to receive the drug sample (block 306). The electronic notification can be sent through scheduled tasks that can be accessed in a secure manner by the prescriber or the prescriber's office personnel. The electronic notification may be sent in other ways as well. The electronic notification can include any or all of the information that the pharmaceutical manufacturer entered upon sending the drug sample to the prescriber.

As the prescriber receives notification that the drug sample is (or shortly will be) en route to the prescriber's office, the prescriber easily can keep track of whether the drug samples he or she is supposed to receive actually are received. This may help minimize drug samples being redirected, for example, during shipping or handling to non-prescriber destinations and may thereby help ensure that drug samples reach their intended prescriber destinations.

Once the drug sample arrives at the prescriber's office, the prescriber confirms receipt of the drug sample (block 308). In a typical implementation, receipt is confirmed by making an entry to this effect at the first user interface device 202 in the prescriber's office. The prescriber or designated office staff uses the first user interface device 202, for example, to access a secure website and takes actions at the website to indicate that the drug sample has been received and that the drug sample delivery matches what was expected to be received. In one example, the secure website presents to the prescriber a list of characteristics (e.g., drug name and strength, quantity, lot number and expiration date) that the drug sample delivery should possess. This list may be generated based on information that was entered by the pharmaceutical manufacturer when the drug sample was being shipped. The secure website can include a user-selectable link prompting the prescriber to confirm that the received drug sample matches the information presented.

In some implementations, the secure website enables the prescriber to make corrections to the information presented if, for example, any of the characteristics (e.g., drug name and strength, quantity, lot number or expiration date) of the received drug sample do not match the information presented at the secure website. If the prescriber corrects one or more of the delivered drug sample characteristics, the system 112 automatically notifies the pharmaceutical manufacturer that sent the drug sample delivery of the correction(s) made.

If the drug samples have machine-readable labels (e.g., bar-codes) with information about the drug samples, then the prescriber or designated office staff can enter the coded information into the system 112 by scanning the label with a scanner coupled to the first user interface device 202 at the prescriber's office. Once the information has been scanned into the system 112, the prescriber or designated office staff is prompted to confirm that scanned information is correct or to enter corrections manually to scanned information as appropriate. The system 112 presents the prescriber with a selectable prompt to confirm that the information in the system (either as scanned or as corrected) is correct and accurately reflects the shipment received. The system 112 then notifies the pharmaceutical manufacturer that the prescriber has received the shipment.

Since the prescriber confirms receipt of drug sample deliveries and the pharmaceutical manufacturers receive receipt confirmation, the prescriber and the pharmaceutical manufacturer easily can keep track of drug sample deliveries. Moreover, any discrepancies between what the pharmaceutical manufacturer believes was sent to the prescriber and what the prescriber indicates was received will be identified in a timely manner so that the cause of the discrepancy can be investigated and corrective actions, if appropriate, can be taken.

When the drug sample is received, inventory data for the drug sample at the prescriber's office is updated (block 310). In a typical implementation, the inventory data for drug samples at a prescriber's office is stored at a local memory storage device 206 within the prescriber's office.

In some implementations, the system 112 is operable to update the inventory data stored in the local memory storage device 206 automatically in response to the prescriber confirming receipt of a particular delivery. If, for example, the prescriber has confirmed receipt by indicating in the system 112 that the characteristics of the delivery match the characteristics entered by the pharmaceutical manufacturer when the delivery was shipped, then, in response to the prescriber's confirmation, the system updates the inventory data based on the information entered by the pharmaceutical manufacturer.

If, on the other hand, the prescriber has made corrections to the information entered by the pharmaceutical manufacturer when confirming receipt, then the system 112 can use the corrected information to update the inventory information.

The system 112 also allows the inventory data to be updated manually when a drug sample shipment is received at a prescriber's office. In those implementations, when a drug sample shipment is received at the prescriber's office, the prescriber or designated office staff can update the inventory data by entering appropriate information with a keyboard, for example, at the first user interface device 202 in the prescriber's office.

Referring again to the method illustrated in FIG. 3, sometime after receiving a drug sample shipment, the prescriber decides to dispense a sample of the drug to a patient (block 312). The prescriber can use the system 112 to manage the drug sample dispensing and can accesses the system's functionality by using a browser to access a website or local software.

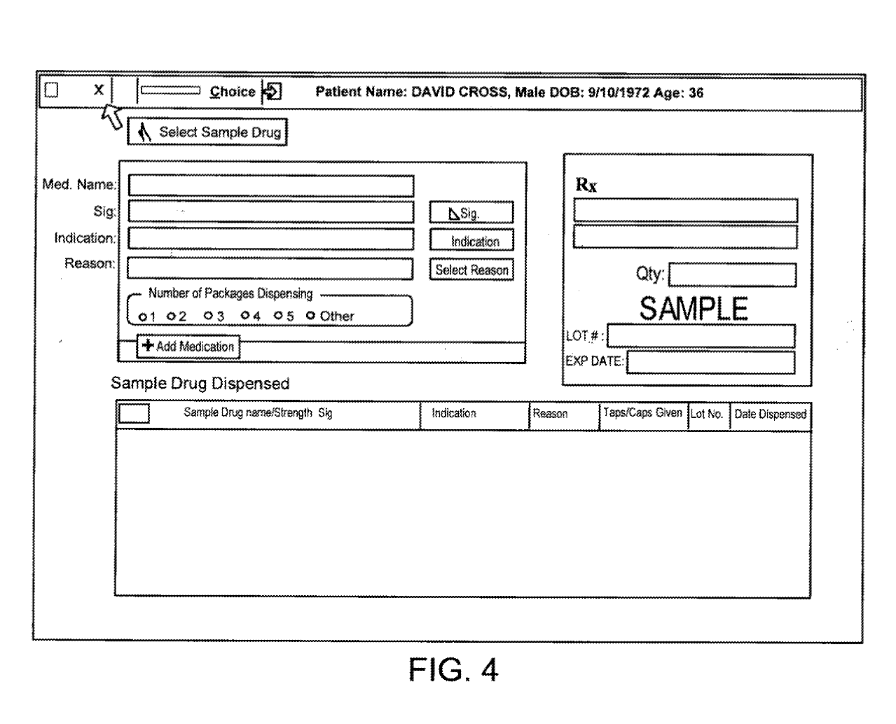

FIG. 4 is an example of a patient-specific main screen that the system presents to the prescriber at the prescriber's first user interface device 202. The illustrated screen and associated system functionality can assist a prescriber in dispensing a drug sample in a safe manner. It also can help facilitate the tracking of drug samples and understanding how the drug samples are being used.

In some implementations, the illustrated screen is accessed by logging into a secure website and specifying a patient's name. In some embodiments, after the prescriber logs in, the system presents the prescriber with a user-selectable list of patient names. The prescriber then selects the patient to whom the prescriber intends to give the drug sample. In the illustrated example, the prescriber would have selected the patient “David Cross,” whose name, gender, date of birth and age appear at the top of the screen. In the illustrated example, the screen identifies David Cross as a male, having a date of birth of Sep. 10, 1972 and being 36 years old.

The upper left section of the screen is for entering data about the drug sample being dispensed, the upper right section of the screen shows a printable label for the drug sample being dispensed, and the lower portion of the screen is a table that builds as the prescriber selects drug samples to be dispensed to the patient. Previous samples dispensed can be found in the patient's medication history.

The upper left section of the screen includes four data entry fields labeled: “Med Name,” “Sig,” “Indication,” and “Reason.” The “Med Name” and a section for indicating the name of the drug are being dispensed as a sample. The “Sig” data entry field is for entering directions for using the drug sample being dispensed. The “Indication” data entry field is for identifying the medical basis for dispensing the drug (e.g., the disease being treated). The “Reason” data entry field is for entering information about why the prescriber has decided to dispense the drug as a sample to the patient.

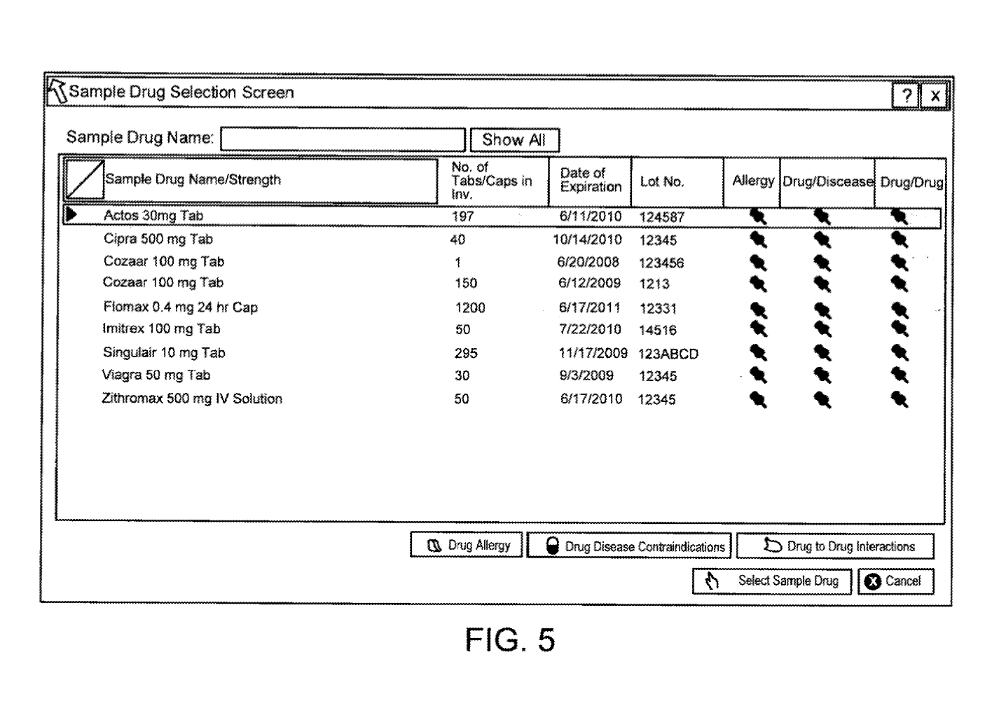

In some instances, the prescriber may wish to enter information manually into each of the data entry fields. Alternatively, the prescriber may use the various buttons provided to access other screens that will help him or her populate the data entry fields. One such button is positioned immediately above the data entry fields on the screen and is labeled “Select Sample Drug.” Selection of the “Select Sample Drug” button causes the system 112 to present to the prescriber a screenshot such as that shown in FIG. 5.

The screenshot of FIG. 5 shows a listing of the drugs that are available at the prescriber's office inventory for dispensing as a sample. The listing includes the drug sample names/strengths, the numbers of tablets/capsules available, expiration dates and lot numbers.

The illustrated screenshot also provides the prescriber with an indication of whether the drug sample being dispensed might adversely affect the patient receiving the drug sample. This information is conveyed, in the illustrated implementation, by virtual thumbtacks that appear under the headings “Allergy,” “Drug/Disease” and “Drug/Drug.” In some implementations, the heading “Therapeutic Duplications” may appear as well. In a typical implementation, the virtual thumbtacks are color-coded to indicate the likelihood and severity of the side effects the sample drug may cause to the patient. Such side effects may result from the drug's interactions with the patient's allergies, a disease the patient has been diagnosed with, or other drugs that the patient is taking. In a particular implementation, for example, a red virtual thumbtack would indicate a high likelihood of serious adverse effects, a yellow virtual thumbtack flag would indicate a moderate likelihood of moderate adverse effects and a green virtual thumbtack would indicate a low likelihood of adverse effects. There are other ways this information can be conveyed to the prescriber.

In some embodiments, the system 112 determines the likelihood and potential severity of possible adverse effects by comparing patient-specific medical history data with data about the various drugs available for dispensing as a sample at the prescriber's office. The patient-specific medical history data can be entered into the system 112 by the prescriber or may be downloaded from a remote patient information database 210, such as the National Patient Health Information Network™ by SureScript-RxHub, LLC. Other information may be obtained from other sources, such as from FirstDataBank. Patient-specific medical history data including, among other things, patient allergies, and diseases that the patient has been diagnosed with may be entered locally by the prescriber or designated office staff.

The patient-specific medical history data can be stored in the prescriber's local memory storage device 206. Each time a patient is scheduled for an appointment with the prescriber, the system 112 updates the information stored in the local memory storage device 206 by sending a request to the patient information database 210 for updated information and downloading any updated information required. In some implementations, the system is set up so that a request is sent to the patient information database 210 for updated information about a patient the evening before the patient's appointment with the prescriber. This way, the information the prescriber will see when dispensing a drug sample to the patient will be as up-to-date as possible.

Referring again to the screenshot of FIG. 5, there are three buttons at the bottom of the screen labeled “Drug Allergy,” “Drug Disease Contraindications” and “Drug to Drug Interactions.” Clicking on the “Drug Allergy” button when one of the listed drug samples is highlighted causes the system 112 to present detailed, patient-specific information about the likelihood and possible severity of adverse effects being caused by the highlighted drug's interactions with the patient's known allergies. Similarly, clicking on the “Drug Disease Contraindications” button when one of the listed drug samples is highlighted causes the system 112 to present detailed, patient-specific information about the likelihood and possible severity of adverse effects being caused by the highlighted drug's interactions with the patient's known diseases. Moreover, clicking on the “Drug to Drug Interactions” button when one of the listed drug samples is highlighted causes the system 112 to present detailed, patient-specific information about the likelihood and possible severity of adverse effects being caused by the highlighted drug's interactions with other drugs the patient is known to be taking. Clicking the “Drug to Drug Interactions” button also causes the system 112 to present detailed, patient-specific information about whether the highlighted drug might duplicate the therapeutic effect of another drug that the patient is known to be taking.

The screenshot in FIG. 5 includes a searching function with a data entry field, labeled “sample drug name” and an activation button, labeled “show all” near the top of the screen. The searching function may be particularly helpful, for example, if the prescriber has a large inventory of drug samples. In that case, the prescriber could simply enter the name of the drug he or she is interested in dispensing and select the “show all” button, which would cause the system 112 to search the list and illustrate only those list entries that match the search term(s) entered.

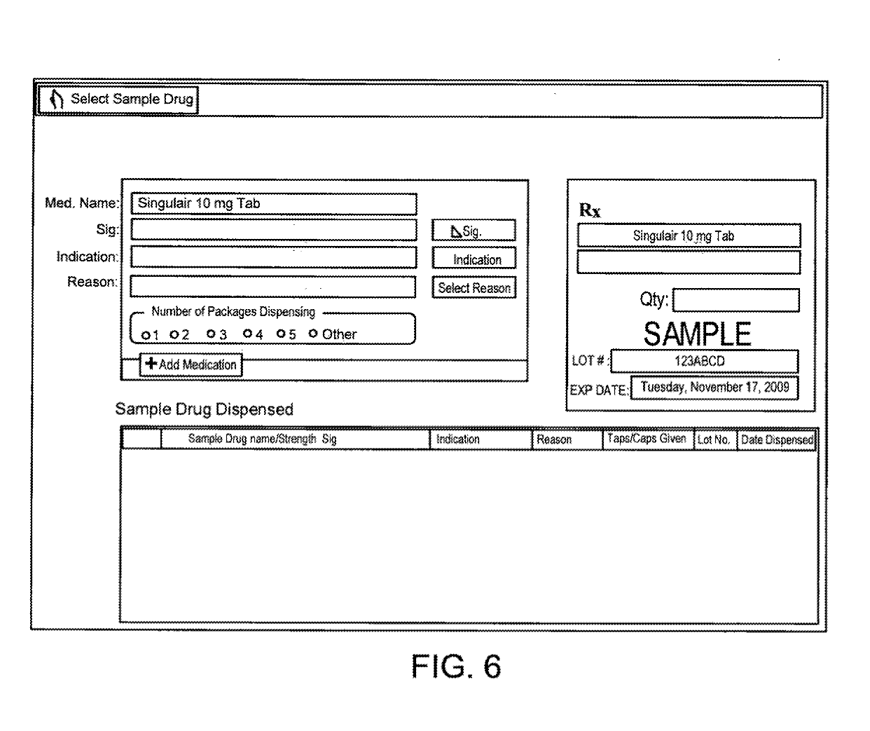

Once the prescriber identifies a listed drug he or she is interested in dispensing, the prescriber can highlight the row in the list that corresponds to the drug and double click or click the “Select Sample Drug” button near the bottom of the screen. This causes the system to return to the main screen and to populate automatically certain information about the drug sample being dispensed. FIG. 6 shows an example of this. Specifically, the screenshot of FIG. 6 shows the main screen that is returned by the system if the prescriber selects the “Singulair 10 mg Tab” line in FIG. 5 and clicks the “Select Sample Drug” button. In the illustrated screenshot, the system 112 has populated the “Med Name” data entry field with “Singulair 10 mg Tab” automatically. The system also has populated the printable label to indicate the drug name/strength (“Singulair 10 mg Tab”) as well as the lot number (“123ABCD”) and the expiration date (“Tuesday, Nov. 17, 2009”). This information is imported from the data shown in FIG. 5, automatically.

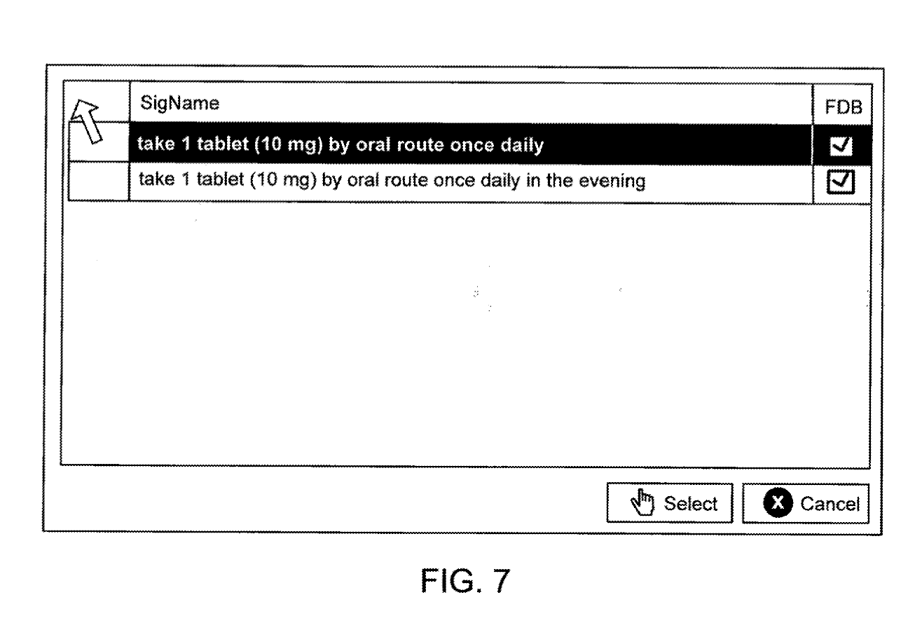

Clicking on the “Sig” button in FIG. 6 causes the system 112 to present the screen of FIG. 7, which includes a list of possible directions for using the selected drug. The designation FDB in the illustrated screenshot refers to the FirstDataBank, Inc. a leading provider of electronic drug information to the health care industry. A checkmark appearing below the FDB heading indicates that the corresponding direction is downloaded from the FirstDataBank database.

In a typical implementation, the system is operable so that the host 218 of FIG. 2 receives periodic (e.g., nightly) updates of data from one or more data sources, such as the database run by FirstDataBank, Inc. The host 218 then may periodically distribute the updated information to the various prescribers' offices.

The prescriber accessing this screen can scroll through the list of recommended directions and highlight the directions he or she thinks are appropriate. Once this is done, clicking the “select” button at the bottom of the page causes the system to return the screenshot of FIG. 6, with the “Sig” data entry field filled in and the corresponding data field in the label portion of the FIG. 6 screenshot filled in as well. If, for example, the “select” button of FIG. 7 were clicked with the list selection highlighted as shown in FIG. 7, or by double clicking the highlighted record then the system would import the entry “take 1 tablet (10 mg) by oral route once daily” into the “Sig” data entry field and into the label portion in the main screen of FIG. 6.

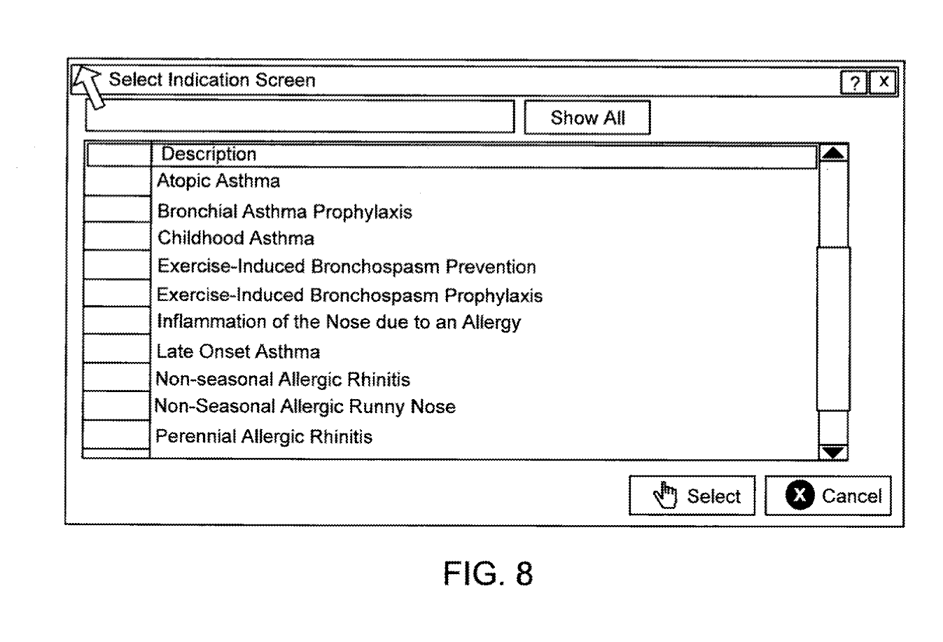

Clicking on the “Indication” button in FIG. 6 causes the system to present the screen shown in FIG. 8, which includes a list of indications for using the sample drug. The prescriber can scroll through the listed indications or can use the search function at the top of the screen to find an appropriate indication. Once an appropriate indication is identified, the prescriber can highlight it and click on the “select” button. This causes the system 112 to return the main screen of FIG. 6 and to populate the corresponding data entry field in the main screen automatically. If, for example, the “select” button of FIG. 8 were clicked with the “Non-Seasonal Allergic Runny Nose” list entry highlighted, the system 112 would return the main screen of FIG. 6 and automatically populate the “Indication” data entry field with “Non-Seasonal Allergic Runny Nose.”

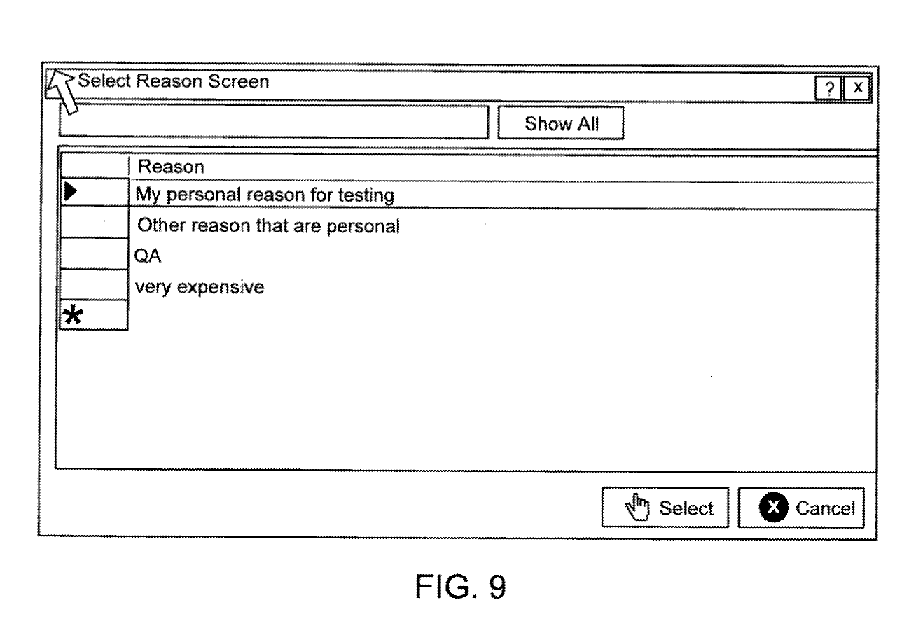

Clicking on the “Select Reason” button in FIG. 6 causes the system 112 to present the screen shown in FIG. 9, which includes a list of reasons for dispensing the sample drug. The prescriber can scroll through the listed reasons or can use the search function at the top of the screen to find an appropriate reason. Once an appropriate indication is identified, the prescriber can highlight it and click on the “select” button. This causes the system 112 to return to the main screen and to populate the corresponding data entry field automatically. If, for example, the “select” button of FIG. 9 were clicked with the “very expensive” list entry highlighted, the system 112 would return the main screen of FIG. 6 and automatically populate the “Reason” data entry field with “very expensive.”

The main screen of FIG. 6 also includes a section that enables the prescriber to specify the number of drug sample packages being dispensed to the patient. The options provided are 1, 2, 3, 4, 5 or other. In a typical implementation, the system 112 stores data indicating how many tablets, for example, there are in each package of a drug sample. Accordingly, in response to the prescriber indicating the number of packages being dispensed, the system populates the “quantity” field in the label portion of the screen. If, for example, the prescriber is dispensing three packages and each package includes two tablets of Singulair, then, in response to the prescriber selecting that three packages are being dispensed, the system would populate the “quantity” field in the label portion of the screen with the number six (6).

The data entry fields in FIG. 6 (e.g., “Sig,” or “Reason”) can be populated by typing information into the data entry field rather than by navigating through the screenshots of FIGS. 7 and 9. In some implementations, the system is adapted so that if information is typed into one or more of the data entry fields, the typed entry is saved and will be presented to the prescriber in one of the screens of FIGS. 7 and 9 when that screen is accessed in the future. If, for example, the only directions in the system 299 for using Singulair 10 mg tablets were: “take 1 tablet (10 mg) by oral route once daily,” but the prescriber wanted the patient to take ½ of tablet by oral route once daily, then the prescriber could type in the corresponding data entry field of FIG. 6 “take ½ tablet by oral route once daily.” In some implementations, the system is adapted to operate so that it saves the newly entered directions. Once saved, the newly entered directions are available for the prescriber to see in the future by accessing the screen of FIG. 7 for the corresponding drug (i.e., Singulair).

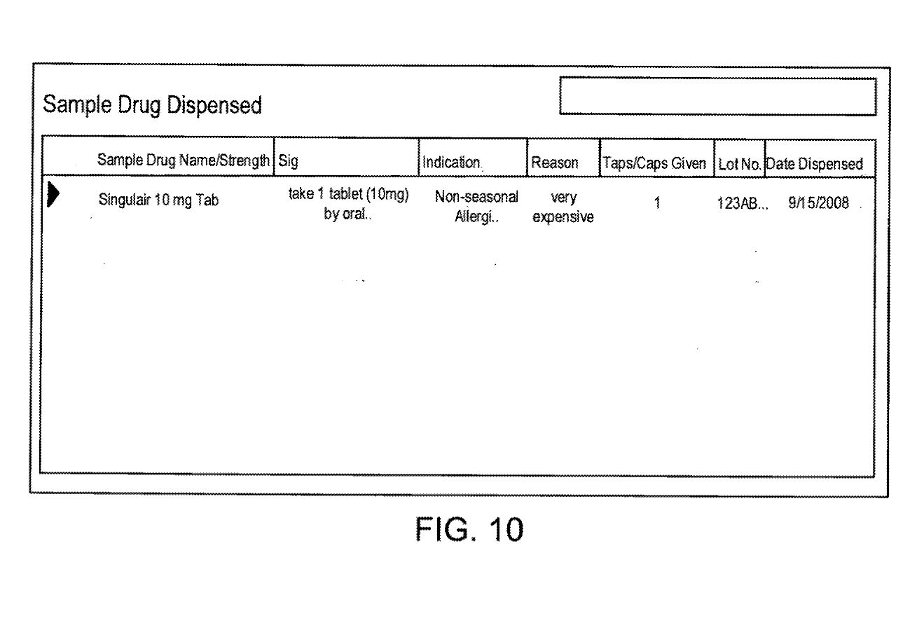

Once the illustrated data entry fields have been populated, clicking the “Add Medication” button causes the system to transfer the information entered to the bottom portion of the screen, with the current date, automatically. An example of this is shown in FIG. 10., which is a partial (bottom section only) view of the main screen in FIG. 6, with the bottom portion populated.

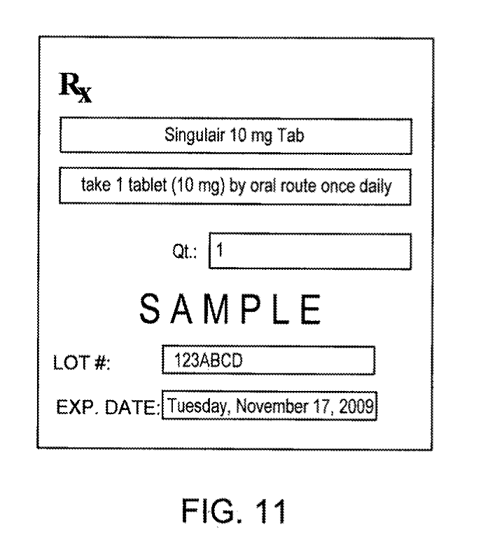

Once the relevant information is transferred to the bottom portion of the screen, a label for the drug sample and associated patient educational material about the drug sample can be printed by prompting the system 112 to do so. The prompting can be implemented in any number of ways, one example of which is by right clicking an area of the screen to access a pop-up menu that includes a printing option. An example of a label that can be printed is shown in FIG. 11.

The patient educational material can include a variety of information about the drug being dispensed to the patient. This information can include, for example, drug uses, safety data, monitoring parameters, recommendations regarding how to use the drug, drug-drug interaction data, drug-disease contraindications and other information that a patient might be interested in learning about the drug being dispensed and that might otherwise be provided to the patient if a pharmacist were involved.

Referring again to FIG. 3, when a drug sample is dispensed, the system 112 updates 316 the stored inventory data for the dispensed drug at the prescriber's office accordingly (block 316). In this way, the system can maintain a relatively up-to-date record of drug sample inventory at the prescriber's office.

In some implementations, the system 112 of FIG. 2 helps prescribers automate certain aspects of inventory maintenance. Specifically, the system 112 enables the prescriber to specify a minimum threshold-level inventory for each drug inventoried at the prescriber's office. As the system 112 maintains a relatively up-to-date record of drug sample inventory at the prescriber's office, the system 112 can recognize when the inventory of a particular drug reaches or drops below the specified minimum (“par”) level.

When the inventory of a particular drug sample does drop below a prescriber-specified minimum level, the system 112 automatically notifies the prescriber, the appropriate pharmaceutical manufacturer and/or the appropriate sales representative that the inventory is low (block 318). In some implementations, the notification sent to the pharmaceutical manufacturer and/or the sales representative includes an order for additional inventory of the drug samples. The notification is sent electronically over the wireless communication network and arrives at the second user terminal device 204 in the appropriate pharmaceutical manufacturer's (or sales representative's) office. In response to receiving this notification, the pharmaceutical manufacturer (or sales representative) automatically can send or bring a previously-agreed amount of samples to the prescriber's office.

In some implementations, the system (via host 218) enables the prescriber to determine readily whether the patient's insurance would cover the drug being dispensed as a sample. The system can obtain this information, for example, from a remote database such as the national patient information network 210 (via 218) or from the prescriber's office's internal records. In a typical embodiment, the system would present visual indicators, such as flags in the screenshot of FIG. 5, to show whether the listed drugs would be covered by insurance.

Referring again to FIG. 2, the data entered by the prescribers, pharmaceutical manufacturers and/or sales representatives in connection with distributing and dispensing drug samples to patients is saved by the system 112. At least some of the data can be stored locally (e.g., in a prescriber's local memory storage device 206 or in a pharmaceutical manufacturer's local memory storage device 208) and at least some of the data can be stored remotely (e.g., in the remote memory storage device 214). The system 112 enables the sharing of this information in a manner that is helpful to each party and to patients, without compromising the patients' privacy (Health Insurance Portability & Accountability Act of 1996-compliant). Specifically, the system 112 enables users to generate 320 reports for the various parties involved in drug sample distribution.

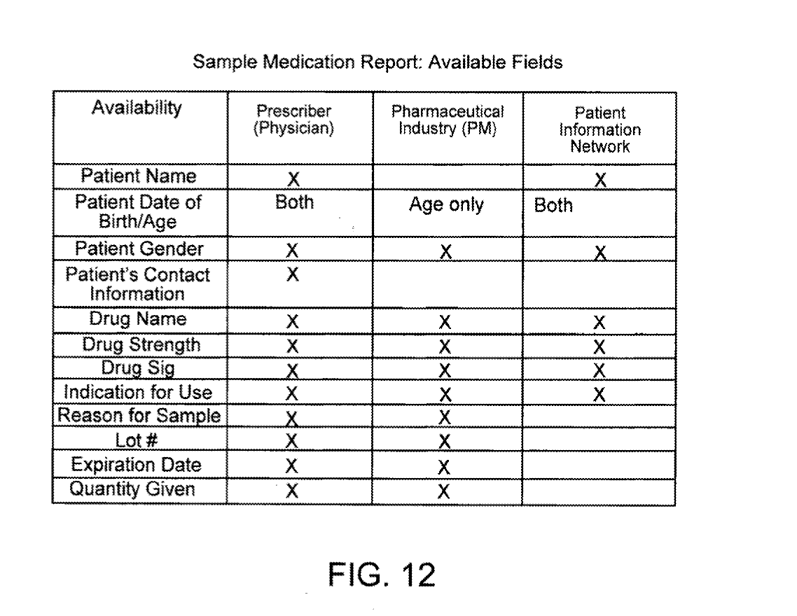

FIG. 12 is a table summarizing some of the data that the various parties can access from the system 112 about a patient and the drug samples dispensed to the patent. The prescriber (“prescriber”) has the most access and can see the patient's name, date of birth/age, gender, contact information, drug name, drug strength, drug sig, indications for use, reason for sample, lot number, expiration date, and quantity given. The pharmaceutical manufacturers (“pharmaceutical industry”) have more restricted access to the patient-related information. For example, as illustrated, the pharmaceutical manufacturers cannot access the patient's name, date of birth or contact information. The pharmaceutical manufacturers, however, can access valuable information about how the drug samples were dispensed as well as the patients' genders and ages. As shown, the system also shares data with the national patient information network 210. More specifically, the national patient information network 210 can receive the patient's name, date of birth, age, gender, the drug name, strength, sig and indication for use.

The system 112 also enables the various parties using the system 112 to generate reports based on the information stored in the system. For example, the system 112 enables prescribers to generate recall reports, black box warning reports and expiration reports. A recall report includes a list of all patients who have received a certain sample drug based on recalled drug name, expiration date, and/or lot number. A black box warning report includes a list of all patients who are taking (or have taken) the drug with the new black box warning. Prescribers may wish to generate a black box report to review listed patients and notify affected patients, if necessary. An expiration report includes a list of drug samples that will expire within a short period of time (e.g., within the next 30 days) so that the prescriber can pull those drugs from stock and remove them from inventory, triggering notification of the pharmaceutical company.

Additionally, the system 112 enables pharmaceutical manufacturers to generate prescriber-specific inventory status reports, accountability reports, sample drug utilization reports, recall or black box warning reports, blinded patient demographic data and longitudinal prescribing data. A prescriber-specific inventory status report indicates the par levels at the various prescribers' offices. An accountability report monitors the accountability of pharmaceutical sales representatives or alternate delivery methods in delivering drug samples to prescriber's office. This may be particularly helpful to pharmaceutical manufacturers trying to comply with the Prescription Drug Marketing Act of 1987. A sample medication utilization report monitors prescriber utilization of target sample medications. A recall report identifies prescribers' offices that have dispensed recalled drug samples. A black box warning report disseminates black box warnings rapidly. Blinded demographic data can include crucial blinded data about patients who have received sample medications (e.g., age, gender, indication for use, reason for dispensing sample). Longitudinal prescribing data links drug sample dispensing with subsequent prescribing of the medication previously given as a sample. The longitudinal prescribing data requires that the prescriber also utilize a related electronic prescribing module where prescribers use the module to generate electronic prescriptions. The system tracks and therefore can link whether a drug dispensed as a sample is later prescribed using the electronic prescribing module.

In some implementations, the system also sends drug sample distribution data to supplement existing medication history information stored in the national patient information database.

In some implementations, the prescriber's office includes a multi-drawer medication cabinet with an automatic dispensing system, such as those available through Med-Dispense, L.P. of Alpharetta, Ga. This can provide an added degree of security in the handling and distribution of the drug samples. Additionally, access to the system 112 may be restricted by requiring entry of user names, passwords and/or utilization of biometric fingerprint scanners.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a computer-readable medium for execution by, or to control the operation of, data processing apparatus. The computer-readable medium can be a machine-readable storage device, a machine-readable storage substrate, a memory device, a composition of matter effecting a machine-readable propagated signal, or a combination of one or more of them. The terms “data processing apparatus” and “computer” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

In a typical implementation, a prescriber may begin using the system by installing (from a disk or by downloading) software to facilitate secure interactions with the host 218. Similarly, a pharmaceutical manufacturer may install software to facilitate secure interactions with the host 218.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a personal digital assistant (PDA). Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described is this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

Although this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, although operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The steps described as being performed by the pharmaceutical manufacturer could similarly be performed by a representative of the pharmaceutical manufacturer and on behalf of the pharmaceutical manufacturer. As such, the term pharmaceutical manufacturer includes the pharmaceutical manufacturer, its employees, consultants and agents and any other representative (e.g., a sales representative) acting on behalf of the pharmaceutical manufacturer.

Similarly, the steps described as being performed by the prescriber could, alternatively, be performed by representatives of the prescriber on behalf of the prescriber. As such, the term prescriber includes the prescriber, his or her employees, consultants and agents and any other representative acting on behalf of the prescriber.

Although particular implementations of the invention have been described, other implementations are within the scope of the claims.

1. A computer-based method comprising:
storing inventory information for one or more drug samples received at a prescriber's office;
presenting a list of drugs available in the inventoried samples; and
automatically updating the stored inventory information for a selected drug in response to an indication from a user that a quantity of the selected drug is being dispensed to a patient as a sample.

2. The computer-based method of claim 1 further comprising:
enabling a pharmaceutical manufacturer to enter details describing a drug sample being sent to the prescriber's office;
enabling the prescriber to confirm receipt of the drug sample; and
in response to the prescriber's confirmation, updating the stored inventory information for the received drug sample.

3. The computer-based system of claim 1 further comprising:
in response to the user's selection of one of the listed drugs, presenting patient specific information regarding the selected drug's suitability for dispensing as a sample.

4. The computer-based method of claim 3 wherein the patient-specific information regarding the selected drug's suitability for dispensing as a sample includes information selected from the group consisting of:
whether the patient has allergies to the selected drug;
whether the selected drug has the potential to cause adverse side-effects to the patient by interacting with other drugs that have been prescribed to the patient;
whether the selected drug is contraindicative to a disease that the patient has been diagnosed with; and
whether administration of the selected drug might represent a therapeutic duplication of another drug that the patient is taking.

5. The computer-based method of claim 3 wherein the patient-specific information regarding the selected drug's suitability for dispensing as a sample comprises whether the selected drug would be covered by the patient's insurance if the selected drug were prescribed to the patient.

6. The computer-based method of claim 1 further comprising:
presenting a user-selectable list of directions for use of the selected drug, wherein the directions have been approved by the Food and Drug Administration for use of the selected drug; and
generating, in response to the user selecting one of the directions for use, a label that includes the selected directions for use and patient specific information (name, address, etc.).

7. The computer-based method of claim 1 further comprising:
in response to a user prompt, printing educational material associated with the selected drug,
wherein the educational material includes material selected from a list consisting of: how to use the selected drug;
the selected drug's potential side effects; and
the selected drug's monitoring parameters.

8. The computer-based method of claim 1 further comprising:
prompting the user to specify a reason why the selected drug is being dispensed as a sample to the patient.

9. The computer-based method of claim 8 wherein prompting the user to specify the reason for dispensing the selected drug as a sample to the patient comprises:
presenting a user-selectable list of reasons for dispensing the selected drug as a sample to the patient,
wherein the list of possible reasons include reasons selected from the group consisting of the following:
to conduct a trial of the selected drug;
to manage an acute condition with the selected drug
to respond to the patient's financial limitations; and
to respond to the patient's request for the selected drug.

10. The computer-based method of claim 1 further comprising:
after a prescriber has dispensed one of the drugs as a sample to the patient and subsequently prescribed a different one of the drugs to the patient, tracking what is later prescribed; and
enabling a manufacturer of the drug dispensed as a sample to access information 10 related to what is later prescribed.

11. The computer-based method of claim 1 further comprising:
prompting the user to enter demographic data for the patient to whom the drug sample is being dispensed; and
enabling a manufacturer of the drug sample being dispensed to access the demographic data associated with the patient in a manner that protects the patient's privacy.

12. The computer-based method of claim 9 further comprising:
generating, in response to a user prompt, a list of patients to whom a particular one of the drugs has been dispensed as a sample, based on drug name, expiration date or lot number.

13. The computer-based method of claim 1 wherein the stored inventory information comprises: drug names, strength of sample drug available quantities of the drugs at the prescriber's office and the drugs' lot numbers and expiration dates.

14. The computer-based method of claim 1 further comprising:
automatically updating the stored inventory information for the selected drug in response to an indication from the user that a quantity of the selected drug is being dispensed to the patient as a sample.

15. The computer-based method of claim 14 further comprising:
automatically identifying when the inventory of one or more of the drug samples has reached or dropped below a pre-defined minimum value.

16. The computer-based method of claim 15 further comprising:
automatically notifying a manufacturer of the drug sample whose inventory has reached or dropped below the pre-defined minimum value that additional inventory should be sent to the prescriber's office.

17. The computer-based method of claim 1 further comprising:
enabling the user to generate a list of drug samples in the prescriber office's inventory based on drug name, expiration date and lot number.

18. A computer-based method comprising:
storing inventory information for one or more drug samples received at a prescriber's office;
presenting a user-selectable list of drugs available in the inventoried samples; and
in response to a user's selection of one of the listed drugs, presenting patient-specific information regarding the selected drug's suitability for dispensing as a sample.

19. The computer-based method of claim 18 wherein the patient-specific information regarding the selected drug's suitability for dispensing as a sample includes information selected from the group consisting of:
whether the patient has allergies to the selected drug;
whether the selected drug has the potential to cause adverse side-effects to the patient by interacting with other drugs that have been prescribed to the patient;
whether the selected drug is contraindicative to a disease that the patient has been diagnosed with;
whether administration of the selected drug might represent a therapeutic duplication of another drug that the patient is taking; and
whether the selected drug would be covered by the patient's insurance if the selected drug were prescribed to the patient.

20. The computer-based method of claim 18 further comprising:
presenting a user-selectable list of directions for use of the selected drug, wherein the directions have been approved by the Food and Drug Administration for use of the selected drug; and
generating, in response to the user selecting one of the directions for use, a label that includes the selected directions for use and patient specific information.

21-42. (canceled)
